(19) United States
(12) Plant Patent Application Publication
(10) Pub. No.: US 2002/0129417 P1
(43) Pub. Date: Sep. 12, 2002

(54) Chrysanthemum named Vyking Orange Dark

(76) Inventor: Arnold Vijverberg, 's-Gravenzande (NL)

Correspondence Address: PARKHURST & WENDEL, 1421 PRINCE STREET, SUITE 210, ALEXANDRIA, VA, 22314, US

(21) Appl. No.: 09/757,634

(22) Filed: Jan. 11, 2001

(51) Int. Cl. A01H 5/00

(52) U.S. Cl. PLT/296

(57) ABSTRACT

A Chrysanthemum plant named VYKING ORANGE DARK characterized by its medium sized blooms with orange ray-florets and brown disc florets.

### RELATED APPLICATIONS

The present application is related to the following plant patent:

| Plant Pat. No. | Filing Date | Title |
|---|---|---|
| | | VYKING |

### BACKGROUND OF THE INVENTION

The new plant of the present invention comprises a new and distinct cultivar of chrysanthemum plant that is a natural occuring sport of a parent chrysanthemum named VYKING. A comparison with Parent chrysanthemum VYKING is also made in this application. The new cultivar was discovered as a whole plant mutation in April 1999 by Nol Vijverberg in a controlled environment (greenhouse) in 's-Gravenzande Holland. The first act of asexual reproduction of VYKING ORANGE DARK was accomplished when vegetative cuttings were taken from the initial selection in June 1999 in 's-Gravenzande Holland.

### SUMMARY OF THE INVENTION

The present invention is a new and distinct variety of chrysanthemum bearing medium sized blooms with orange ray-florets and brown disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

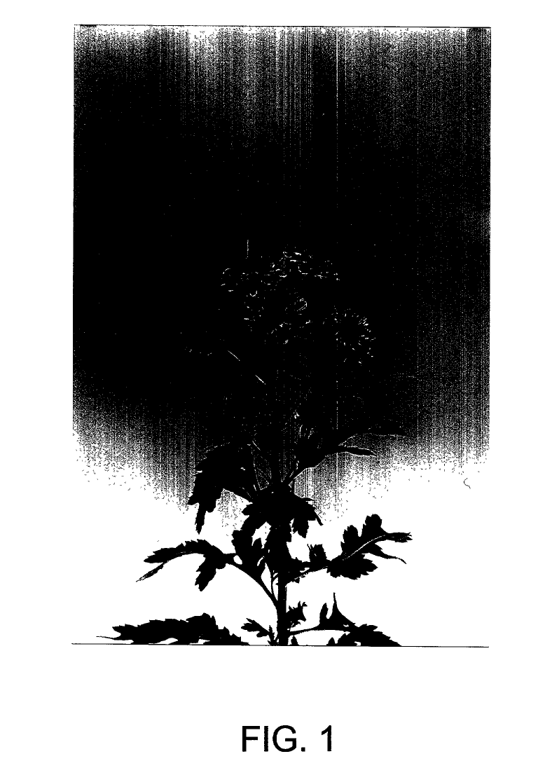

FIG. 2 shows the various stages of bloom of the cultivar.

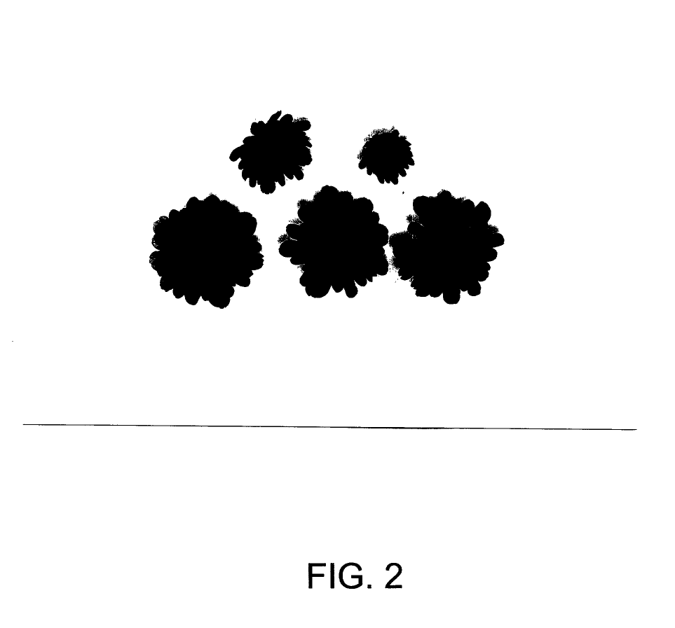

FIG. 3 shows the foliage of the cultivar.

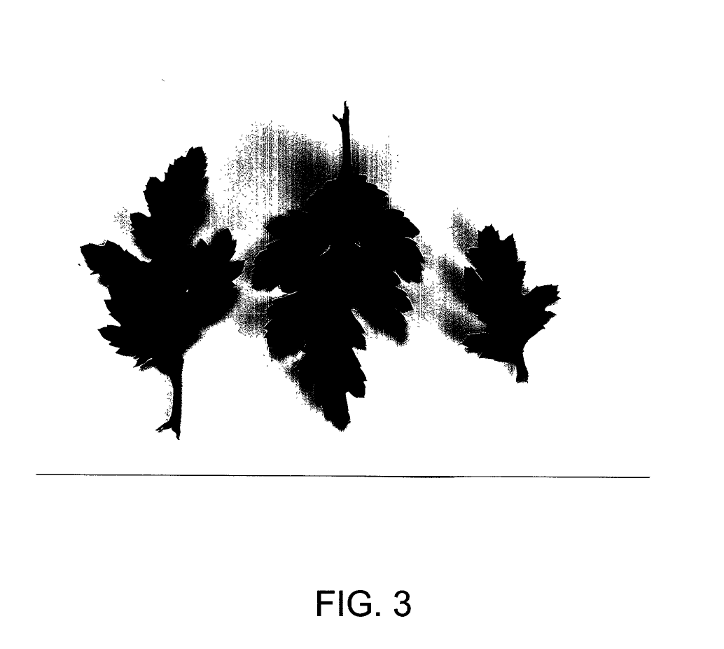

### DESCRIPTION OF THE INVENTION

This new variety of chrysanthemum is of the botanical classification Dendranthema Grandiflora. The observations and measurements were gathered from plants grown in a greenhouse in 's-Gravenzande Holland in a photo-periodic controlled crop. The photo-periodic response time in this crop was 52 days. This new variety produces medium sized blooms with orange ray-florets and brown disc-florets blooming on the plant for 3 weeks. This new variety of chrysanthemum has been found to retain its distinctive characteristics throughout successive propagations however the phenotype may vary significantly with variations in environment such as light intensity and temperature. To show the phenotype as described VYKING ORANGE DARK may be planted without assimilation lightning between week 50 and 40 under greenhouse conditions in Holland. With assimilation lightning (minimum level 2500 lux) it may be planted year round under greenhouse conditions in Holland.

The variety VYKING ORANGE DARK is a natural mutant of VYKING. When VYKING and VYKING ORANGE DARK are being compared the following differences and similarities are noticed: The difference of VYKING and VYKING ORANGE DARK is (1): flower color and (2) flower form. All other characteristics of VYKING and VYKING ORANGE DARK are the same.

(1) Flower color: VYKING ORANGE DARK is a color mutant of VYKING bearing orange blooms instead of yellow blooms.

(2) Flower form: The ray florets of VYKING ORANGE DARK are more folded and more concave as the ray florets of VYKING.

The following is a description of the plant and characteristics that distinguish VYKING ORANGE DARK as a new and distinct variety.

The color designations are taken from the plant itself. Accordingly, any discrepancies between the color designations and the colors depicted in the photographs are due to photographic tolerances.

Botanical Description of

| CULTIVAR VYKING | | |
|---|---|---|
| ORANGE DARK | | VYKING |
| Bud | | |
| Size | large cross-section +/− 1.5 | |
| | cm height +/− 1.2 cm | |
| Outside Color | Greyed-orange 169D | Yellow-orange |
| | | 15 B |
| Bloom | | |
| Size | Small | |
| Fully Expanded | 4-4.5 cm | |
| Borne | Upper portion single flower | |
| | per peduncle lower portion | |
| | plural flowers per peduncle | |
| number of blooms per | Average of 8 | |
| single stem | | |
| Peduncle length | Near the top +/− 6 cm, near | |
| | the middle +/− 12 cm, near | |
| | the bottom +/− 16 cm | |
| Peduncle lateral shoot | Medium | |
| attachment | | |
| Peduncle lateral shoot angle | Medium, 30° | |
| Form | Daisy | |
| Performance | 14 days | |
| (blooming period) | | |
| Color | | |
| Center of the flower | Immature greyed-orange | |
| (disc-florets) | 166A | |
| | mature greyed-orange 165A | |
| Color of upper surface of | Greyed-orange 169C | Yellow 12A |
| the majority of the | | |
| ray-florets | | |
| Color of the lower surface | Greyed-orange 169D | Yellow 8A |
| of the majority of the | | |
| ray-florets | | |
| Tonality from Distance | A medium greyed-orange | |
| | daisy with a typical brown | |
| | center | |
| Discoloration | Some to greyed-orange | |
| | 169D | |
| Ray florets | | |
| Texture | Upper and under side | |
| | smooth | |
| Number | 55-65, three rows | |
| Cross-section | Concave, one keel | |
| Longitudinal axis of | Straight to incurved | Straight |
| majority | | |
| Shape of tip | Rounded, sometimes | |
| | dentate | |
| Fragrance | Typical chrysanthemum | |
| Disc Diameter | Medium, +/− 1.5 cm | |
| (when visible) | | |
| Reproductive Organs | | |
| Stamen (present in | Yellow-green, thick, +/− 4 | |
| disc florets only) | mm in length | |
| Pollen | None | |
| Styles (present in both ray | Yellow-green, thick | |
| and disc florets) | | |
| Style Length | +/−5 mm | |
| Stigmas | Yellow-green | |
| Stigma Width | +/−2 mm | |
| Ovaries | Enclosed in calyx | |
| Plant | | |
| Form | Spray mum meant for erect | |
| | culture. Herbaceous | |
| Growth | Short to medium | |
| Height | +/−100 cm | |
| Stem Color | Yellow-green 146A | |
| Stem Strength | Strong | |
| Stem Brittleness | Present | |
| Stem Anthocyanin | Present (mainly at the base) | |
| Coloration | | |
| Internodes | Medium (2.5-3 cm) | |
| Flowering Response | 56 days | |
| (photo-periodic controlled | | |
| crop, no natural growing) | | |
| Foliage | | |
| Color | Upper side Yellow-green | |
| | 147A | |
| | under side Yellow-green | |
| | 147B | |
| Size | Large; length +/− 12 cm, | |
| | width +/7 cm | |
| Quantity (number per | 22-26 | |
| single stem) | | |
| Shape | Egg shaped | |
| Texture | Fleshy | |
| Ribs and Veins | Ribs and veins well | |
| | developed | |
| Edge | Crenated | |
| Shape of Base of Sinus | Round | |
| Between Lateral Lobes | | |
| Margin of Sinus Between | Converging | |
| Lateral Lobes | | |
| Shape of Base | Truncate and asymmetric | |
| Apex | Mucronate | |

I claim:

1. A new and distinct variety of chrysanthemum plant as described and illustrated.
